(19) United States
(12) Patent Application Publication
(10) Pub. No.: US 2009/0221899 A1
(43) Pub. Date: Sep. 3, 2009

(54) MINIMALLY INVASIVE RECTAL BALLOON APPARATUS WITH PRESSURE RELIEVING LUMEN AND ANAL VERGE DILATION COLLAR

(76) Inventor: John ISHAM, Houston, TX (US)

Correspondence Address: EGBERT LAW OFFICES, 412 MAIN STREET, 7TH FLOOR, HOUSTON, TX, 77002, US

(21) Appl. No.: 12/412,017

(22) Filed: Mar. 26, 2009

Related U.S. Application Data
(63) Continuation-in-part of application No. 11/966,544, filed on Dec. 28, 2007.
Application No. 11/966,544 is a continuation-in-part of application No. 11/933,018, filed on Oct. 31, 2007.
Application No. 11/933,018 is a continuation-in-part of application No. 11/623,702, filed on Jan. 16, 2007.

(51) Int. Cl. A61M 36/06; A61M 29/02

(52) U.S. Cl. 600/407; 606/192; 600/3

(57) ABSTRACT

A rectal balloon apparatus has a shaft with a fluid passageway extending therethrough. A balloon is affixed over an end of the shaft such that the fluid passageway communicates with an interior of the balloon. A collar is affixed around the shaft which is suitable for dilating a patient's anal verge. The collar is a color distinct from the colors of the balloon and the shaft. The collar has a first section hingedly connected to a second section so as to allow the collar to be selectively affixed to the shaft. The collar has an outer diameter that is roughly twice an outer diameter of the shaft. The collar can be snap-fitted around the shaft.

### CROSS-REFERENCE TO RELATED U.S. APPLICATIONS

The present application is a continuation-in-part of U.S. application Ser. No. 11/966,544, filed on Dec. 28, 2007, entitled “Minimally Invasive Rectal Balloon Apparatus with Pressure Relieving Lumen,” presently pending. U.S. application Ser. No. 11/966,544 is a continuation-in-part of U.S. application Ser. No. 11/933,018, filed on Oct. 31, 2007, entitled “Minimally Invasive Rectal Balloon Apparatus”, presently pending. U.S. application Ser. No. 11/933,018 is a continuation-in-part of U.S. application Ser. No. 11/623,702, filed on Jan. 16, 2007, entitled “Minimally Invasive Rectal Balloon Apparatus”, presently pending.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The present invention relates to an apparatus for facilitating performance of diagnostic and therapeutic treatment of disease. Particularly, the present invention relates to rectal balloons that are used for immobilizing the region surrounding the prostate during pre-treatment simulation and target localization, as well as during the delivery of radiation therapy to treat prostate cancer. More particularly, the present invention relates to collars that are used to facilitate the positioning of the rectal balloon by providing a collar that accommodates the anal verge.

2. Description of Related Art Including Information Disclosed Under 37 CFR 1.97 and 37 CFR 1.98.

Treatment of prostate cancer using radiation therapy is difficult due to the prostate's position near radiation-sensitive tissues, and is further complicated by prostate motion. Adenocarcinoma of the prostate commonly occurs in the posterior portion of the prostate gland which is in very close proximity to the rectal wall. To plan external beam radiation treatment, urethrograms, CT scans and magnetic resonance imaging (MRI) have all been used to visually localize the prostate, as well as the normal critical structures in the surrounding area.

U.S. Pat. No. 5,476,095, issued on Dec. 19, 1995 to Schnall et al., describes an insertable pickup probe for use in providing diagnostic MRI images. The pickup probe in its preferred embodiment is for use in imaging the male prostate and comprises an elongated shaft supporting an inflatable patient interface balloon at its distal end. The interface balloon comprises an inner balloon and an outer balloon, between which a receiving coil is positioned. A lumen for air supply is provided in the shaft for expanding the inner balloon against the outer balloon to place the receiving coil in close proximity to the area of interest in order to provide MRI images.

Typically, the planning of radiation therapy for the treatment of prostate cancer involves the patient undergoing a CT-based simulation scan of the pelvis to determine the location of the prostate gland. In the simulation phase, the patient is placed on CT equipment that is preferably similar to the radiation treatment equipment (except that it does not generate the high energy radiation beam). The simulation equipment is positioned to simulate the delivery of the sequence of treatment beams prescribed by the treating oncologist. Normally, during the simulation procedure, CT images are acquired. These CT images allow the oncologist to locate the position of the tumor and help to facilitate the composition of a radiation treatment plan. This treatment plan delineates the positions of the radiation equipment components for delivery of the treatment beams.

During the actual treatment phase, the patient is placed in the same position on the treatment equipment as in the simulation scans. Radiation-emitting devices are generally known and used for radiation therapy in the treatment of patients. Typically, a radiation therapy device includes a gantry, which can be swiveled around a horizontal axis of rotation in the course of a therapeutic treatment. A linear accelerator is located in the gantry for generating a high-energy radiation beam for therapy. During treatment, the radiation beam is provided by this equipment and is delivered to the patient at the precise location as delineated by the physician during simulation. A further feature of radiation therapy involves portal images, which are commonly used in radiation therapy to verify and record the patient tumor location. Portal images include manual (film) and electronic images (EPI) taken before and/or after the treatment.

During external beam radiation therapy, radiation is directed to the target prostate which is near the rectal wall. A misdirected radiation beam may perforate the rectal wall causing radiation proctitus (rectal bleeding). This toxicity is related to the total radiation dose prescribed and the volume of the anterior rectal wall receiving a high radiation dose. A major factor limiting radiation oncologists' attempts to reduce the volume of the anterior rectal wall receiving a high radiation dose is the position of the prostate gland as well as the intrinsic motion up to 5 millimeters in the anterior to posterior direction caused by rectal peristalsis. Accordingly, oncologists generally will add a margin to the radiation field in order to ensure that the entire prostate gland receives the prescription dose. This margin is typically on the order of 5 to 15 millimeters. As a consequence, lower doses of radiation may need to be used so as not to overexpose radiation sensitive structures. However, this may lead to inadequate radiation treatment and a higher probability of local cancer recurrence.

U.S. Patent Publication No. 2003/0028097, published on Feb. 6, 2003 to D′Amico et al., describes an immobolizer probe system and method. This system has an insertable probe for immobilizing a region of interest during staging and radiation therapy thereof. In particular, this device uses a balloon having a rectangular cross section connected to a shaft. The shaft extends to an end of the balloon so as to allow fluid flow through an interior of the shaft and into the balloon so as to selectively inflate the balloon once the balloon is installed into the rectal cavity. The balloon, shaft and handle are bonded together so that they move radially as a single unit when torque is applied. A syringe is provided which connects the shaft and serves as an air pump to deliver a volume-limited amount of air to the air lumen of the shaft to the balloon. A stop cock is provided to maintain the air within the balloon.

One of the problems with the subject of U.S. Patent Publication No. 2003/0028097 is the discomfort associated installing the rectal balloon within the rectal cavity. In particular, a relatively sturdy and wide diameter shaft is connected to a relatively large thick-walled balloon. Because the balloon is not supported by anything other than by the shaft, the balloon is formed of a relatively rugged and thick material. Because of the relatively large size of the shaft and the thick material of the rectangular-cross section balloon, the installation of the rectal balloon creates a large amount of discomfort for a patient. It is often difficult for the medical personnel to know exactly how far within the rectum the balloon has been installed. It is difficult to achieve a standardized and fixed position of the balloon during each and every use. The medical personnel must generally approximate the desired position of the balloon within the rectal cavity. As such, a need has developed whereby the rectal balloon can be formed of a minimal diameter shaft and of a balloon of relatively thin material.

When the rectal balloon of U.S. Patent Publication No. 2003/0028097 is in an inflated condition, the outer surface is generally round. As such, the prostate will tend to balance on the curved surface rather than properly seating thereon. Since seating is important for proper use, this device requires that the physician approximate a seated position rather than providing any feedback of the seated position. When the balloon is in a curved inflated condition, the prostate will have a tendency to slide to one side of the balloon. As such, a need developed to provide a rectal balloon that retains the prostate in a proper seated position when the balloon is in a fully inflated condition.

U.S. application Ser. No. 11/623,702, filed on Jan. 16, 2007 to the present inventor, describes a rectal balloon apparatus which has a shaft with a fluid passageway extending therethrough, a balloon affixed over an end of the shaft such that the fluid passageway communicates with the interior of the balloon, and a sleeve slidably affixed over the shaft. This sleeve is movable between a first position overlying the balloon and a second position away from the balloon. The sleeve has an aperture at an end thereof such that the balloon extends outwardly of this aperture.

U.S. application Ser. No. 11/933,018, filed on Oct. 31, 1997 to the present inventor, additionally describes the balloon as having a laterally flat surface thereon when inflated. There is a longitudinal groove extending along the laterally flat surface. Together, the laterally flat surface and the groove enhance the seating of the prostate on the balloon. Additionally, the application describes a valve assembly on the end of the shaft opposite the balloon. A fluid introducing means is removably attached to the valve assembly to introduce fluid into the valve assembly and move the balloon from the non-inflated condition to the inflated condition.

A very important consideration when treating patients using radiation therapy is that the proper dose of radiation reaches the treatment site. This is very important whether the treatment method utilizes implanted radiation seeds, fluids or external beams of radiation. Excessive dosing of the patient can lead to severe side effects. A proper treatment plan should deliver an adequate amount of radiation to the treatment site while minimizing the dose delivered to the surrounding tissues. Various patents have issued that aid a treating professional in monitoring the dosage received at the treatment site.

U.S. Pat. No. 6,963,771, issued on Nov. 8, 2005 to Scarantino et al., describes a method, system and implantable device for radiation dose verification. The method includes (a) placing at least one wireless implantable sensor in a first subject at a target location; (b) administering a first dose of radiation therapy into the first subject; (c) obtaining radiation data from at least one wireless implantable sensor; and (d) calculating a radiation dose amount received by the first subject at the target location based on the radiation data obtained from the wireless sensor during and/or after exposure to the first administered dose of radiation to determine and/or verify a dose amount of radiation delivered to the target location.

U.S. Pat. No. 7,361,134, issued on Apr. 22, 2008 to Rozenfeld et al., teaches a method of determining the dose rate of a radiation source including locating three or more detectors in the vicinity of a source. Each of the detectors provides an output indicative of the amount of radiation received from the source and determines the location of the source from at least some of the detector outputs.

A significant cause of patient discomfort associated with rectal balloons of the prior art is the buildup of gas pressure when the balloon is inserted into the rectum. This buildup of gas pressure can also affect the ability to properly seat the balloon in a desired position. Various studies, including one outlined in “Reduction of Prostate Motion by Removal of Gas in Rectum During Radiotherapy,” by Ogino et al. (Int. J. Radiation Oncology Biol. Phys., Vol. 72, No. 2) show that prostate and seminal vesicle motion can be substantially reduced by the removal of rectal gas during and before radiation therapy. The study shows the beneficial effects of reduction of motion of the prostate and seminal vesicle. In the past, rectal gas removal had been accomplished by a patient or physician inserting his finger into the patient's rectum. Another way to accomplish the gas removal is by prescribing laxatives to a patient prior to treatment. Many patients may find that removing rectal gas using their fingers is uncomfortable. Additionally, patients needing such treatment often exhibit symptoms of diarrhea, which makes prescription of laxatives improper. As such, a need developed to provide a rectal balloon apparatus that allows for the release of bodily gas pressure.

U.S. application Ser. No. 11/966,544, filed on Dec. 28, 2007 to the present inventor, describes a rectal balloon apparatus that has a shaft with a fluid passageway extending therethrough. The shaft communicates with the interior of the balloon. To achieve the goal of rectal gas removal, the apparatus also has a lumen that extends along the shaft so as to allow bodily gas pressure to pass through it to the atmosphere outside of the patient.

During experimentation with the balloons described above, it was found that insertion of the balloon was relatively comfortable. However, the anatomical region interior to the balloon consisting of the anal verge, was not addressed. The anal verge region can be included in the treatment margin and therefore the benefits of tissue sparing through dilation are an advantage as well. This region is difficult to address with a balloon due to the tighter forces applied by the muscles of the anus. As such, a need has developed to provide a rectal balloon apparatus that maintains or dilates the anal verge during treatment and before radiation therapy.

In certain circumstances, a physician may or may not wish to open the anal verge in order to enhance patient comfort and to enhance the ability to properly position the rectal balloon. As such, a need has developed to allow the physician to apply an aperical collar in a desired location on the shaft so as to address the anal verge region during or prior to treatment. Conventional rectal balloon devices have relatively wide diameter rigid shafts. These wide diameter rigid shafts will dilate the anal verge region but are too restrictive for the balloon to perform properly.

It is an object of the present invention to provide a rectal balloon apparatus which is easy to use and easy to install.

It is another object of the present invention to provide a rectal balloon apparatus that has a mechanism to relieve bodily gas pressure prior to or during the inflation of the balloon.

It is another object of the present invention to provide a rectal balloon apparatus that has an aperical collar mechanism which the treating professional can utilize to dilate the anal verge before radiation therapy and/or during treatment.

It is another object of the present invention to provide a rectal balloon apparatus whereby the position of the balloon can be easily ascertained by medical personnel.

It is a further object of the present invention to provide a rectal balloon apparatus which maximizes the comfort to the patient.

It is a further object of the present invention to provide a rectal balloon apparatus which has better holding stability when inflated.

It is a further object of the present invention to provide a rectal balloon apparatus which has a flexible shaft.

It is another object of the present invention to provide a rectal balloon apparatus which can be inflated to several configurations for different medical purposes.

It is another object of the present invention to provide a rectal balloon apparatus which enhances the ability to properly seat the prostate on a flat surface of the rectal balloon.

It is yet another object of the present invention to provide a rectal balloon apparatus which reduces prostate and seminal vesicle motion during treatment.

It is a further object of the present invention to provide a rectal balloon apparatus which avoids potential allergic reactions.

It is a further object of the present invention to provide a collar for a rectal balloon apparatus that can be easily applied to the shaft in an area adjacent the balloon at the discretion of the physician.

It is still a further object of the present invention to provide a rectal balloon apparatus which is easy to manufacture and relatively inexpensive.

These and other objects and advantages of the present invention will become apparent from a reading of the attached specification and appended claims.

### BRIEF SUMMARY OF THE INVENTION

The present invention is a rectal balloon apparatus that comprises a shaft having a fluid passageway extending therethrough and a balloon affixed over an end of the shaft such that the fluid passageway communicates with an interior of the balloon. The balloon is movable from a non-inflated condition to an inflated condition. The balloon has a first end and a second end. The first end is proximal to the end of the shaft and the second end is at an opposite end of the balloon. The apparatus also has a collar affixed around the shaft adjacent the second end of the balloon. The collar has a diameter greater than the diameter of the shaft. The collar may be affixed over the shaft at the treating professional's discretion to address the anal verge area.

In the present invention, the collar is formed of a rigid material and has a diameter approximately twice the diameter of the shaft. The collar has a length of approximately one inch, and a color distinct from the colors of the balloon and the shaft.

The balloon has a generally laterally flat surface thereon when it is in the inflated condition. The balloon is movable from the inflated condition to a second inflated condition. In the second inflated condition, the balloon has both a laterally flat surface and a bulbous surface. There is a groove formed in the laterally flat surface that extends centrally longitudinally along a portion of the laterally flat surface. A radiation sensing means is positioned within the balloon for sensing the amount of radiation received at the treatment site.

The apparatus also has a lumen which extends along the length of the shaft so as to have one end open at or beyond the first end of the balloon and a second end that opens along the shaft at a position remote from the balloon. The lumen is formed and extends interior of the shaft. A splitting means is formed on the shaft remote from the balloon which allows the lumen to pass exteriorly of the shaft. A fluid introducing means is in fluid communication with the fluid passageway of the shaft and moves the balloon from the non-inflated condition to the inflated condition.

The rectal balloon apparatus of the present invention has a collar that can be affixed to the shaft selectively by the physician so as to accommodate the anal verge. The collar has a first section and a second section that can be connected together around the shaft. The first section is hingedly connected to the second section. Each of the first section and the second section have a semi-circular shape with an inner diameter approximately equal to the diameter of the shaft. One of the first and second sections has a receptacle therein at an end opposite the hinge. The other of the first and second sections has a pin connector at the end opposite the hinge. The pin can be snap-fitted into the receptacle so that the sections are joined together and tightly fitted onto the shaft in a location adjacent the balloon. As such, the collar allows the physician the discretion to selectively apply the aperical collar in order to address the anal verge region. As such, the collar can facilitate comfortable insertion and removal of the rectal balloon apparatus.

### BRIEF DESCRIPTION OF THE SEVERAL VIEWS OF THE DRAWINGS

FIG. 1 is a side elevational view, partially transparent, which shows the rectal balloon apparatus in an uninflated condition.

FIG. 2 is a side elevational view of the rectal balloon apparatus of the present invention in the first inflated condition.

FIG. 3 is a side view, partially transparent, of the balloon of the rectal balloon apparatus in the first inflated condition.

FIG. 4 is a top view of the inflated balloon as used in the rectal balloon apparatus of the present invention.

FIG. 5 is a side view, partially transparent, of the balloon of the rectal balloon apparatus in the second inflated condition.

FIG. 6 is an isolated view, partially transparent, of the splitting means of the rectal balloon apparatus of the present invention.

FIG. 7 is a side view of the balloon of the rectal balloon apparatus of the present invention showing the various layers of the balloon.

FIG. 8 is an isolated view showing the valve assembly of the rectal balloon apparatus of the present invention.

FIG. 9 is an isolated view of the collar as used on the rectal balloon apparatus of the present invention.

### DETAILED DESCRIPTION OF THE INVENTION

Referring to FIG. 1, there is shown the rectal balloon apparatus 10 in accordance with the preferred embodiment of the present invention. The rectal balloon apparatus 10 includes a shaft 12 having a fluid passageway extending therethrough. A balloon 14 is affixed over the end 16 of the shaft 12. The balloon 14 is shown in an uninflated condition. The fluid passageway of the shaft 12 can communicate with the interior of the balloon 14.

The shaft 12 is a generally longitudinal shaft which has the fluid passageway extending through the center thereof. The shaft 12 is made of a flexible material. A valve assembly 22 is affixed to the shaft 12 opposite the balloon 14. The valve assembly 22 includes a control knob 28. Turning the control know 28 serves to close the valve assembly 22 so as to selectively allow the fluid to pass into the shaft 12. A port 30 allows the valve assembly 22 to be connected to a supply of the fluid. When the valve assembly 22 is opened by the rotation of the control knob 28, the fluid will flow through the valve assembly 22, through the interior passageway of the shaft 12, and into the interior of the balloon 14. The valve assembly 22 can then be closed so as to maintain the inflated configuration of the balloon 14. When the procedure is finished and the fluid needs to be removed from the balloon 14, the valve assembly 22 can then be opened so as to allow for the release of fluid therethrough.

Between the valve assembly 22 and the balloon 14 is situated the splitting means 20. Although not shown in detail in FIG. 1, the shaft 12 contains both the fluid passageway for inflating and deflating the balloon 14, and a lumen 90 for allowing gas pressure to escape from beyond the end of the balloon 14. The tip 24 of the lumen 90 can be seen extending past the end of the balloon 14. When rectal gas enters the tip 24 of the lumen 90, it flows through the lumen 90 and escapes through the lumen port 21 on the splitting means 20. As shown in greater detail below, the splitting means 20 splits the lumen 90 from the fluid passageway of the shaft 12.

The opposite end 16 of the shaft 12 nearly contacts the end 32 of the balloon 14. The end 16 is suitably curved or dome-shaped so as to allow the shaft 12 to facilitate the introduction of the balloon 14 into the rectal cavity. The shaft 12 has indicia 34 formed therealong. It can be seen that the indicia 34 has numerical references associated therewith. These numerical references are indicative of the distance that the balloon 14 has been inserted into the rectum. As such, the indicia 34 provide a clear indication to the medical personnel of the desired location of the rectal balloon 14.

The balloon 14 is welded to the shaft 12 at the point 26. As shown in FIG. 1, there is a collar 18 which covers the weld at point 26. The collar 18 is constructed of a hard material and locked over the shaft 12. The collar may have a hinge and a locking mechanism. The collar 18 is approximately one inch in length and has an outer diameter suitable for dilating the anal verge when the rectal balloon is inserted. The diameter of the collar 18 should be roughly twice the diameter of the shaft 12. Here, the shaft 12 is approximately two tenths of an inch in diameter and the diameter of the collar 18 should be four tenths of an inch or greater. The dilation of the anal verge by the collar 18 is important in that it allows the patient greater comfort during removal of the balloon after treatment. The collar 18 is of a color distinct from the color of the shaft 12 and the balloon 14 to allow the treating professional to better determine the position of the collar 18.

The anal verge region can be included in the treatment area and dilation of the area can prove advantageous. Dilation is difficult to accomplish with the use of a balloon due to the strong forces applied by the muscles of the anus. Other rectal balloon apparatus of the prior art use a thick and rigid shaft. The prior art shaft design can accomplish the goal of dilation of the anal verge, but such a shaft makes for an invasive and uncomfortable insertion. The minimal shaft diameter of the present invention allows for minimally invasive and comfortable insertion. Should the treating professional desire to address the anal verge area, the collar 18 may be affixed or clamped over the shaft 12. Thus, the present invention accomplishes anal verge dilation without sacrificing comfort.

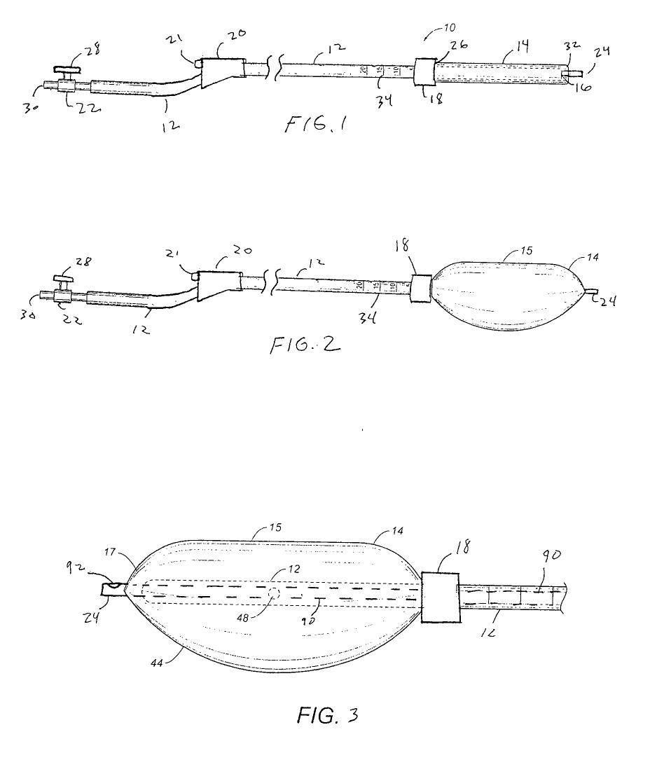

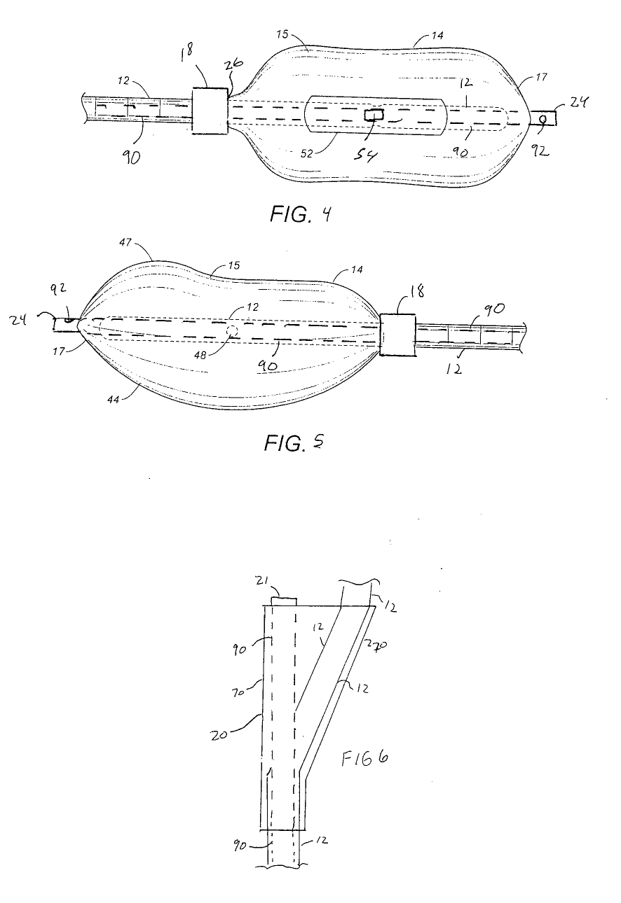

FIG. 2 illustrates the apparatus 10 after the apparatus has been installed within the rectum. The fluid can be introduced through the valve assembly 22 and through the interior passageway of the shaft 12 so as to inflate the balloon 14. The balloon 14 has a seating area 15 so that the prostate can be properly positioned thereon. The balloon 14 has a head portion 17 adjacent the tip of the balloon 14 opposite the shaft 12. When the balloon 14 is installed and inflated, the prostate will reside on the flat surface 15 in a seated position. The head portion 17 will abut the tip of the prostate. After the procedure has been completed, the balloon 14 can be deflated and easily pulled outwardly of the rectum in its deflated condition. Using balloons of the prior art (not having the collar 18), the anal verge would constrict around the shaft 12, making removal difficult.

FIG. 3 is an isolated view of the balloon 14 as inflated to a first inflated condition. In this first condition, the balloon 14 has a central seating area 15, a head portion 17 and a bottom portion 44. The central seating area 15 has a lateral flatness when inflated for the prostate to rest upon. The first inflated condition is formed when approximately 100 cc of fluid are introduced into the balloon 14. The lateral flatness of the seating area 15 will prevent the prostate from sliding to one side or the other. The bottom portion 44 is rounded and contacts the rectal wall. The head portion 17 is generally V-shaped so as to facilitate easier insertion of the balloon 14. The material of the balloon 14 is formed of a non-latex material so as to avoid allergic reactions. The shaft 12 is shown extending into the interior of the balloon 12. A hole 48 is formed in the shaft 12 through which the balloon 14 is filled with fluid. The hole 48 is formed generally centrally within the balloon 14.

Importantly, in FIG. 3, it can be seen that the lumen 90 is contained within the fluid passageway of the shaft 12. The lumen 90 is represented by the large-dashed line interior of the lines representing the shaft 12. As shown, the lumen 90 extends beyond the end of the shaft 12 and through the surface of the balloon 14. The tip 24 of the lumen 90 is exterior of the balloon 14. The tip 24 of the lumen 90 has a hole 92 through which rectal gas passes. When the rectal gas enters the hole 92, it runs through the lumen 90, through the splitting means 20 and into the atmosphere through the lumen port 21. Additionally, it can be seen that the collar 18 has a diameter greater than that of the shaft 12 such that it can dilate the anal verge a suitable amount.

FIG. 4 is a top view of the balloon 14 from the side of the balloon 14 which engages with the prostate. Central seating area 15 is shown as having a groove 52 formed thereon. The groove 52 is generally rectangularly-shaped and engages with the tip of the prostate and reduces lateral motion. The central seating area 15 and the groove 52 enhance the holding stability of the balloon 14 of the present invention. In FIG. 4, it can also be seen that head portion 17 of the balloon 14 is generally V-shaped. This shape makes insertion of the balloon 14 into the rectum easier for medical personnel and more comfortable for the patient. The balloon 14 has a thermally welded bond 26 connecting it to the shaft 12, over which the collar 18 is affixed. Additionally, FIG. 4 shows the radiation sensing device 54 placed within the groove 52. The radiation sensing device can be one known in the prior art (as described above) and is used to remotely determine the amount of radiation received at a treatment site.

FIG. 5 is an isolated view of the balloon 14 as inflated to a second inflated condition. In the second inflated condition, the balloon 14 has a bulbous portion 47 formed adjacent to the head portion 17. The balloon also has a laterally flat seating area 15. The bulbous portion 47 can be utilized in certain conditions to better isolate the prostate. Generally, the bulbous portion 47 will be formed when at least 140 cc of fluid are introduced into the balloon 14.

FIG. 6 is an isolated view, partially transparent, of the splitting means 20. The splitting means 20 has a plastic enclosure 70. The lumen 90 and shaft 12 enter into the plastic enclosure 70 at the bottom end of the splitting means 20. The lumen 90 is represented by a dashed line and the shaft 12 by a solid line. As shown, the two lines are split, with the lumen 90 splitting outwardly of the shaft 12. The lumen 90 extends to the lumen port 21 and the gas within exits to the atmosphere. The shaft 12 extends upwardly and exits the splitting means 20 towards the valve assembly 22. The system is fluid-tight, and at no point does the rectal gas within the lumen 90 mix with the fluid introduced into the shaft 12 at the valve assembly 22.

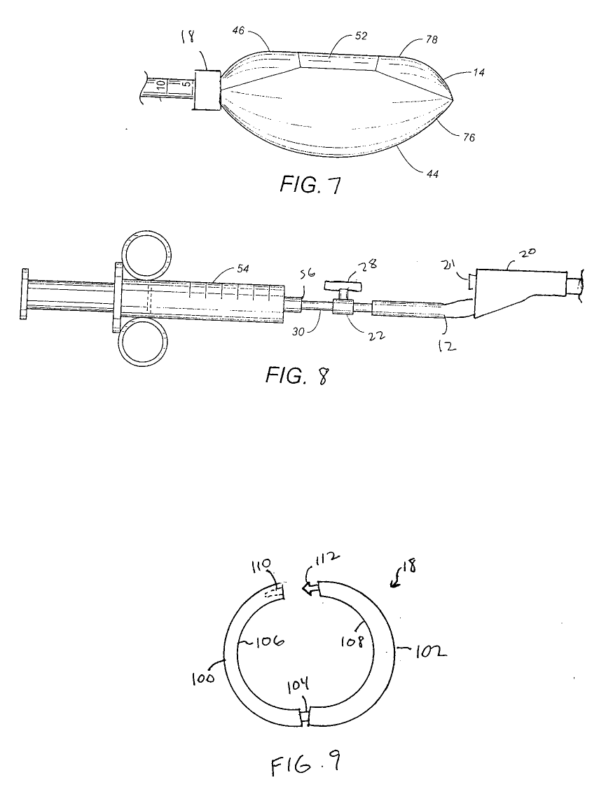

FIG. 7 shows a cross-sectional side view of the balloon 14 showing the plurality of layers which form the balloon 14. A bottom layer 76 forms the bottom portion 44 of the balloon 14. A top layer 78 forms the upper portion, including central seating area 15 and the groove 52, of the balloon 14. A middle layer 80 extends between the bottom layer 76 and the top layer 78. The middle layer 80 is connected to the top layer 78 at the groove 52.

FIG. 8 shows an isolated view of the shaft 12 of the rectal balloon apparatus 10 at an end opposite the balloon 14. The valve assembly 22 is connected at one end to the shaft 12 and at the opposite end to a fluid introducing device 54. In this case, the fluid introducing device 54 is a syringe. The syringe 54 serves as a removable handle for the rectal balloon apparatus 10. An end portion 56 of the syringe 54 is removably connected to the port 30 of the valve assembly 22. When the valve assembly 22 in an open position, fluid can travel from the syringe 54 into the shaft 12 and to the balloon 14. With the balloon 14 inflated, the valve assembly 22 may be closed and the syringe 54 removed. The syringe 54 can be reconnected to the valve assembly 22 prior to removal of the balloon 14 from the rectum to aid in removing the fluid from the balloon 14.

FIG. 9 is an isolated view of the collar 18 as used in the rectal balloon apparatus 10 of the present invention. As can be seen, the collar 18 has a first section 100 and a second section 102 with a hinge 104 connecting sections 100 and 102. Each of the sections 100 and 102 has a generally semi-circular shape. The hinge 104 is connected to an end of first section 100 and an end of second section 102. The first section 100 has a curved inner diameter 106 that has a radius approximately equal to a radius of an outer diameter of the shaft 12. The second section 102 has a curved inner diameter 108 approximately equal to the radius of the outer diameter of the shaft 12. The first section 100 has a receptacle 110 formed on an end thereof opposite hinge 104. The second section 102 has a pin connector 112 formed at an end thereof opposite the hinge 104. The sections 100 and 102 can pivot relative to hinge 104 so as to open wide enough to allow the shaft 12 to pass through a space between the receptacle 110 and pin connector 112. The sections 100 and 102 can then be closed together around the shaft 12 such that the pin connector 112 is engaged within receptacle 110. As such, the collar 18 can be tightly fit onto the shaft 12 so as to fix the position of the collar 18 on the shaft 12. The collar of the present invention can function as an add-on device that can be applied at the physician's discretion. The collar 18 can be applied prior to any insertion of the rectal balloon apparatus 10 or during the insertion process.

The foregoing disclosure and description of the invention is illustrative and explanatory thereof. Various changes in the details of the illustrated construction can be made within the scope of the present claims without departing from the true spirit of the invention. The present invention should only be limited by the following claims and their legal equivalents.

I claim:

1. A rectal balloon apparatus comprising:
a shaft having a fluid passageway extending therethrough;
a balloon affixed over an end of said shaft such that said fluid passageway communicates with an interior of said balloon, said balloon movable between an inflated condition and a non-inflated condition, said balloon having a first end and a second end, said first end being proximal said end of said shaft, said second end being at an opposite end of said balloon; and
a collar affixed around said shaft, said collar being adjacent said second end of said balloon, said collar having a diameter greater than a diameter of said shaft.

2. The apparatus of claim 1, said collar being formed of a rigid material.

3. The apparatus of claim 1, said collar having a diameter approximately twice a diameter of said shaft.

4. The apparatus of claim 1, said collar having a length of approximately one inch.

5. The apparatus of claim 1, said collar having a color distinct from colors of said shaft and said balloon.

6. The apparatus of claim 1, said collar comprising:
a first section having an inner diameter approximately equal to an outer diameter of said shaft; and
a second section having an inner diameter approximately equal to said outer diameter of said shaft, said first second being affixed to said second section.

7. The apparatus of claim 6, each of said first and second sections having a generally semi-circular shape.

8. The apparatus of claim 6, further comprising:
a hinge connected to one end of said first section and to one end of said second section.

9. The apparatus of claim 8, said first section having a receptacle at an end opposite said hinge, said second section having a pin connector at an end opposite said hinge, said pin connector engaged in said receptacle.

10. The apparatus of claim 1, further comprising:
a radiation sensing means positioned within said balloon, said radiation sensing means for sensing an amount of radiation received at a treatment site adjacent said balloon.

11. The apparatus of claim 1, further comprising:
a lumen extending along the length of said shaft so as to have a first end opening at or beyond said first end of said balloon and a second end opening along said shaft at a position remote from said balloon.

12. A rectal balloon apparatus comprising:
a shaft having a fluid passageway extending therethrough;
a balloon affixed over an end of said shaft such that said fluid passageway communicates with an interior of said balloon, said balloon movable between an inflated condition and a non-inflated condition, said balloon having a first end and a second end, said first end being proximal said end of said shaft, said second end being at an opposite end of said balloon; and
a collar affixed around said shaft, said collar being adjacent to said second end of said balloon, said collar having a diameter greater a diameter of said shaft, said collar comprising:
a first section having an inner diameter approximately equal to an outer diameter of said shaft; and
a second section having an inner diameter approximately equal to said outer diameter of said shaft, said first second being affixed to said second section.

13. The apparatus of claim 12, each of said first and second sections having a generally semi-circular shape.

14. The apparatus of claim 13, further comprising:
a hinge connected to one end of said first section and to one end of said second section.

15. The apparatus of claim 15, said first section having a receptacle at an end opposite said hinge, said second section having a pin connector at an end opposite said hinge, said pin connector engaged in said receptacle.

16. The apparatus of claim 12, said first section having an outer diameter approximately twice a diameter of said shaft, said second section having an outer diameter approximately twice the diameter of said shaft.

17. A collar for application to a shaft of a rectal balloon apparatus comprising:
a first section having an inner diameter approximately equal to an outer diameter of the shaft; and
a second section hingedly connected to said first section, said second section having an inner diameter approximately equal to the outer diameter of the shaft, each of said first and second sections having a generally semi-circular shape, said first section being engageable with said second section.

18. The collar of claim 17, said first section having a receptacle at an end opposite said hinge, said second section having a pin connector at an end opposite said hinge, said pin connector engaged in said receptacle.

19. The collar of claim 17, each of said first and second sections having an outer diameter approximately twice the outer diameter of the shaft.

20. The collar of claim 17, each of said first and second sections having a length of approximately one inch and being formed of a rigid material, each of said first and second sections having a color that is different than a color of the shaft.
